SEQUENCE LISTING (5 sequences)

SEQ ID NO: 1 - skipped

SEQ ID NO: 2 - DNA, 5 bp, Artificial Sequence
  Shine-Dalgarno (chemically synthesized)
ggagg

SEQ ID NO: 3 - DNA, 33 bp, Artificial Sequence
  Primer (chemically synthesized)
ggaggcaaccatggatgcacacaagagtgaagg

SEQ ID NO: 4 - DNA, 25 bp, Artificial Sequence
  Primer (chemically synthesized)
ccgtcgacgtagagaagtccgtatt

SEQ ID NO: 5 - DNA, 27 bp, Artificial Sequence
  Primer (chemically synthesized)
gcccatggtaaaatcttggtttattta

(19) United States
(12) Patent Application Publication
(10) Pub. No.: US 2007/0067862 A1
(43) Pub. Date: Mar. 22, 2007

(54) Chloroplast transgenic approach to express and purify human serum albumin, a protein highly susceptible to proteolytic degradation

(75) Inventor: Henry Daniell, Winter Park, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: University of Florida Research Foundation, Inc., Orlando, FL (US)

(21) Appl. No.: 10/563,189

(22) PCT Filed: Jul. 3, 2003
(86) PCT No.: PCT/US03/21158

§ 371 Date: Aug. 9, 2006

(51) Int. Cl. A01H 1/00; C12N 15/82

(52) U.S. Cl. 800/278; 435/468

(57) ABSTRACT

Production of human serum albumin (HSA) in prokaryotic systems has not been successful to date because HSA is highly susceptible to proteolytic degradation. Production in plants has not yielded enough protein to be cost-effective. The instant invention overcomes this by producing HSA in plant plastids at high levels.

### FIELD OF THE INVENTION

This application relates to the field of genetic engineering of plant plastid genomes, particularly chloroplast, vectors for transforming plastids, transformed plants, progeny of transformed plants, and to methods for transforming plastid genomes and plants to generate Human Serum Albumin. This application further relates to regulatory elements, which enhance the expression of biopharmaceutical proteins that are highly susceptible to proteolytic degradation. Further this application relates to the creation of proteolytically stable recombinant biopharmaceutical proteins.

### BACKGROUND

The availability of recombinant human proteins has revolutionized the use of therapeutically valuable proteins in clinical medicine. Plants offer a suitable alternative to microbial or animal expression of biopharmaceutical proteins because of their inexpensive production costs and absence of human pathogens. However, there are some limitations. In particular, expression of human proteins in nuclear transgenic plants has been disappointingly low, e.g. human serum albumin 0.02% of total soluble protein (tsp), human Interferon-b 0.000017% of fresh weight, human epidermal growth factor 0.001% of tsp and erythropoietin 0.0026% of tsp (Daniell et al., 2001d). Therefore, it is important to increase levels of expression in order to exploit plant production of pharmacologically important proteins. As an alternative to nuclear expression, the plastid, and more specifically the chloroplast transgenic approach has been developed as an effective tool for the expression of biopharmaceutical proteins in plants (Daniell and Dhingra, 2002; Daniell et al., 2001a,b; DeGray et al., 2001; Guda et al., 2000.

The plastids of higher plants are an attractive target for genetic engineering. Plant plastids (chloroplasts, amyloplasts, elaioplasts, etioplasts, chromoplasts, etc.) are the major biosynthetic centers that, in addition to photosynthesis, are responsible for production of industrially important compounds such as amino acids, complex carbohydrates, fatty acids, and pigments. Plastids are derived from a common precursor known as a proplastid and thus the plastids present in a given plant species all have the same genetic content. In general, plant cells contain 500-10,000 copies of a small 120-160 kilobase circular plastid genome, each molecule of which has a large (approximately 25 kb) inverted repeat. Thus, it is possible to engineer plant cells to contain up to 20,000 copies of a particular gene of interest which potentially can result in very high levels of foreign gene expression. In addition, plastids of most plants are maternally inherited. Consequently, unlike heterologous genes expressed in the nucleus, heterologous genes expressed in plastids are not pollen disseminated. As a result, a trait introduced into a plant plastid will not be transmitted to wild-type relatives.

After the first demonstration of a protein based polymer expression with varied medical applications (Guda et al., 2000), transgenic chloroplasts have been shown to express very small antimicrobial peptides without fusion proteins (DeGray et al., 2001), assemble functional oligomers with disulphide bonds of the cholera toxin b-subunit (Daniell et al., 2001b), and express a monoclonal antibody with coordinated expression and assembly of heavy and light chain with proper folding and formation of disulphide bridges (Daniell et al., 2001a), suggesting that adequate redox environment or required chaperonins are present within chloroplasts.

Expression of functional human somatotropin in transgenic tobacco chloroplasts established that chloroplasts are capable of proper folding of human proteins with disulphide bonds. The ability to express multiple genes in a single transformation event (Daniell and Dhingra, 2002; De Cosa et al., 2001), accumulation of exceptionally large quantities of foreign proteins (De Cosa et al., 2001), successful engineering of tomato chromoplasts for high level transgene expression in fruits (Ruf et al., 2001), or other edible parts, such as carrots (Kumar et al. 2003), coupled to hyper-expression of vaccine antigens (Daniell et al., 2001b), and the use of plant derived antibiotic free selectable markers (Daniell et al., 2001c), augur well for oral delivery of edible vaccines and biopharmaceuticals that are currently beyond the reach of those who need them most. In addition, chloroplast genetic engineering is an environmentally friendly approach, offering containment of transgenes and a solution to gene silencing and position effect encountered in nuclear transgenic plants (Bogorad, 2000; Daniell and Dhingra, 2002; Daniell et al., 2002; Daniell, 2002).

Human Serum Albumin (HSA) is the most widely used intravenous protein and is prescribed in multi-gram quantities to replace blood volume in trauma and in various other clinical situations (Peters, 1995). HSA is a monomeric globular prepro-protein whose mature form consists of a single polypeptide chain of 585 amino acids (SEQ ID NO:1) (66.5 kDa with 17 disulphide bonds). The annual world need for biopharmaceutically acceptable HSA exceeds 500 tons, representing a market value of more than $1.5 billion. To date, HSA has been produced primarily by the fractionation of blood serum. Lack of glycosylation facilitates production of functional HSA in prokaryotic systems.

The human serum proteins albumin (ALB), .alpha.-feto-protein (AFP) and vitamin D binding protein (VDB) are known to be members of a multigene ALB family. All three proteins are found in serum where they mediate the transport of a wide variety of ligands. ALB binds fatty acids, amino acids, steroids, glutathione, metals, bilirubin, lysolecithin, hematin, prostaglandins and pharmaceuticals (for review, see 1). AFP binds fatty acids, bilirubin and metals. VDB binds vitamin D and its metabolites as well as fatty acids, actin, C5a and C5a des Arg.

In addition to their transport capabilities, ALB family proteins possess a wide assortment of other functional activities. ALB is the main contributor to the colloid oncotic pressure of plasma, acts as a scavenger of oxygen-free radicals and can inhibit copper-stimulated lipid peroxidation, hydrogen peroxide release, and neutrophil spreading. AFP has been implicated in the regulation of immune processes and VDB can act as a co-chemotactic factor for neutrophils and as an activating factor for macrophages.

The serum levels of ALB family proteins are also known to be responsive to various pathological conditions. ALB is a negative acute phase protein (17) whose levels decrease in times of stress. AFP levels are elevated in women carrying fetuses with certain developmental disorders and in individuals with hepatocarcinoma, teratocarcinoma, hereditary tyrosinemia or ataxia-telangiectasia. VDB levels are decreased in patients with septic shock or fulminant hepatic necrosis.

ALB family members also have significant structural similarities. Homology has been observed at the primary amino acid sequence level and there is also a well-conserved pattern of Cys residues which predicts similar secondary structures. ALB family genes have similar exon/intron organizations and all have been mapped to human chromosome 4 within the region 4q11-q22.

Human “Afamin” (abbreviated as “AFM”) is a serum protein with a molecular weight of 87000 daltons. It shares strong similarity to albumin family members and has the characteristic pattern of disulfide bonds observed in this family. In addition, the gene maps to chromosome 4 as do other members of the albumin gene family. Thus, AFM is the fourth member of the albumin family of proteins. AFM cDNA was stably transfected into Chinese hamster ovary cells and recombinant protein (rAFM) was purified from conditioned medium. Both rAFM and AFM purified from human serum react with a polyclonal antibody that was raised against a synthetic peptide derived from the deduced amino acid sequence of AFM. It is expected that AFM will have properties and biological activities in common with ALB, AFP, and VDB.

Although the HSA gene and cDNA have been expressed in a wide variety of microbial systems, including E. coli (Latta et al., 1987), Bacillus subtilis (Saunders et al., 1987), Saccharomyces cerevisiae (Quirk et al., 1989), Kluyveromyces (Fleer et al., 1991) or Pichia pastoris (Ohtani et al., 1998), no system is yet commercially feasible. Sijmons et al. (1990) made the first reported attempt to express HSA in transgenic plants, but very low expression levels were attained (0.02% Total Soluble Protein “tsp”). However, HSA could not be detected if expressed in the cytoplasm, suggesting that HSA, like many biopharmaceutical proteins, is not stable in this compartment, due to high susceptibility of the protein to proteolytic degradation.

A 10-fold increase in HSA accumulation has been reported recently by nuclear transformation of potato plants and targeting the HSA to the tuber apoplast (Farran et al., 2002). Estimates by industry, however, suggest that the cost-effective yield for biopharmaceutical production is 0.1 mg of HSA per gram of fresh weight (Farran et al., 2002). The aforementioned is one example of the problems faced in the art for the production of a cost-effective yield of a recombinant biopharmaceutical protein. In addition, good recombinant systems are still not available for many human proteins that are expensive to purify or highly susceptible to proteolytic degradation. It is known that traditional purification of biopharmaceuticals proteins using columns accounts for 30% of the production cost and 70% of the set up cost (Petrides et al., 1995). Proteolytic degradation is another serious concern for industrial bioprocessing. The increasing production of proteins in heterologous hosts through the use of recombinant DNA technology has brought this problem into focus; heterologous proteins appear to be more prone to proteolysis (Enfors, 1992). Recombinant proteins are often regarded by a cell as foreign and therefore degraded much faster than most endogenous proteins (Rozkov et al., 2000). Proteolytic stability of recombinant proteins is a significant factor influencing the final yield. In view of these limitations, the Applicant has developed a more efficient method of recombinant biopharmaceutical protein, such as HSA production, which may be used as a model system to enrich or purify biopharmaceutical proteins from transgenic plants, which are highly susceptible to proteolytic degradation.

### SUMMARY OF THE INVENTION

In one preferred aspect of this invention, Human Serum Albumin (HSA) accounts for 60% of the total protein in blood serum and it is the most widely used intravenous protein in a number of human therapies. HSA, however, is currently extracted only from blood because of a lack of commercially feasible recombinant expression systems. HSA, like many biopharmaceutical proteins, is highly susceptible to proteolytic degradation in recombinant systems and is expensive to purify. Expression of HSA in transgenic chloroplasts using Shine-Dalgarno sequence (SD), which usually facilitates hyper-expression of transgenes, resulted only in 0.02% HSA in total protein (tp). Modification of HSA regulatory sequences using chloroplast untranslated regions (UTRs) resulted in hyper-expression of HSA (up to 11.1% tp), compensating for excessive proteolytic degradation. This is the highest expression of a biopharmaceutical protein in transgenic plants and 500-fold greater than previous reports on HSA expression in transgenic leaves. Of course higher yields are still possible and the expression reported in this example merely shows one example of the level of HSA expression. Electron micrographs of immunogold labeled transgenic chloroplasts revealed HSA inclusion bodies, which provided a simple method for purification from other cellular proteins. HSA inclusion bodies could be readily solubilized to obtain a monomeric form using appropriate reagents. The regulatory elements used in this Application provide a model system for enhancing expression of foreign proteins that are highly susceptible to proteolytic degradation and provide advantages in purification, when inclusion bodies are formed.

### BRIEF DESCRIPTION OF THE DRAWINGS

FIG. 1(a) is a schematic view of HSA vector constructs, pLDAsdHSA, and pLDApsbAHA.

FIG. 1(b) is a schematic view of the 0.91 kb DNA flanking the cassette, and the 0.75 kb DNA fragment containing the HSA coding region. The 0.81 kb fragment (P1) flanking the cassette and the 0.75 kb fragment containing the HSA coding regions (P2) were used as probes for Southern Blot analysis.

FIG. 1(c) and 1(d) shows Southern Blot analysis using P1(1c) and P2(1d) as probes. Lane 1 shows untransformed DNA; Lane 2 and 3 show plants transformed with pLDAsdHSA; Lanes 4 and 5 show plants transformed with pLDApsbAHSA. Lanes 2 and 4 show plants for the first generation (T∘) and lanes 3 and 5 show (T1) generation.

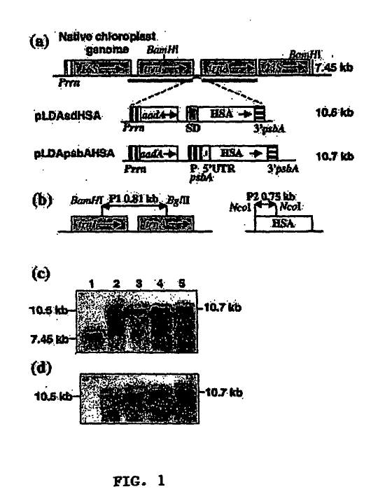

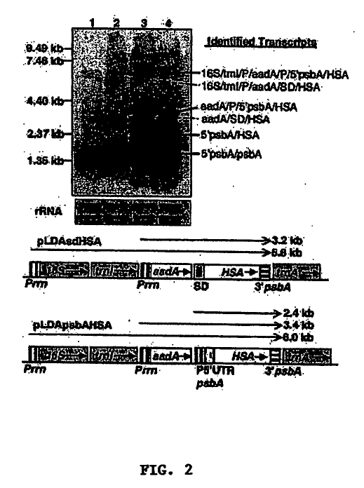

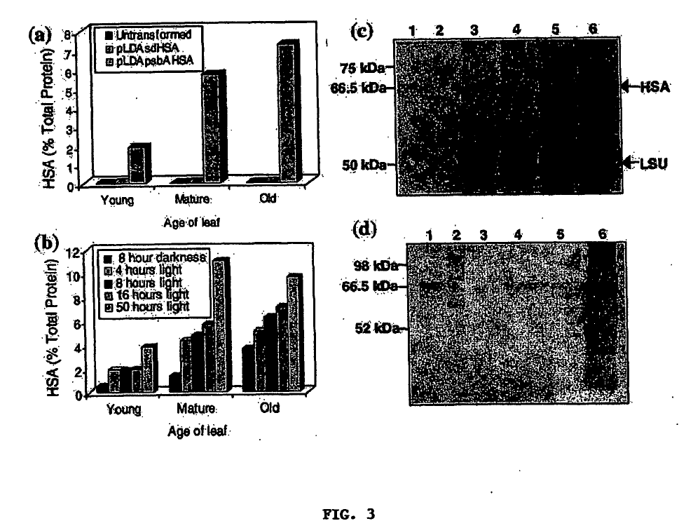

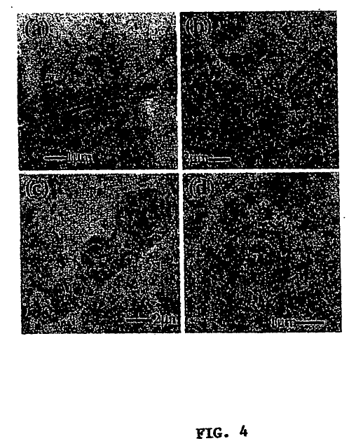

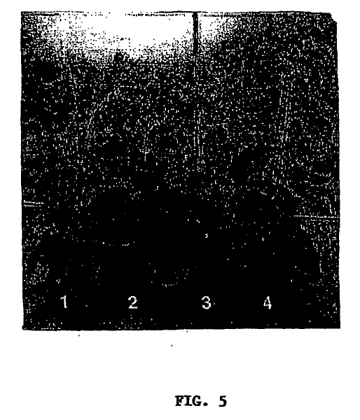

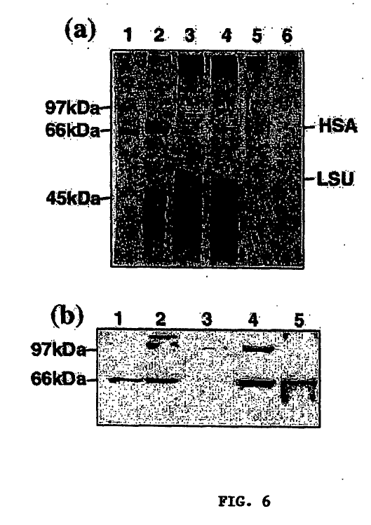

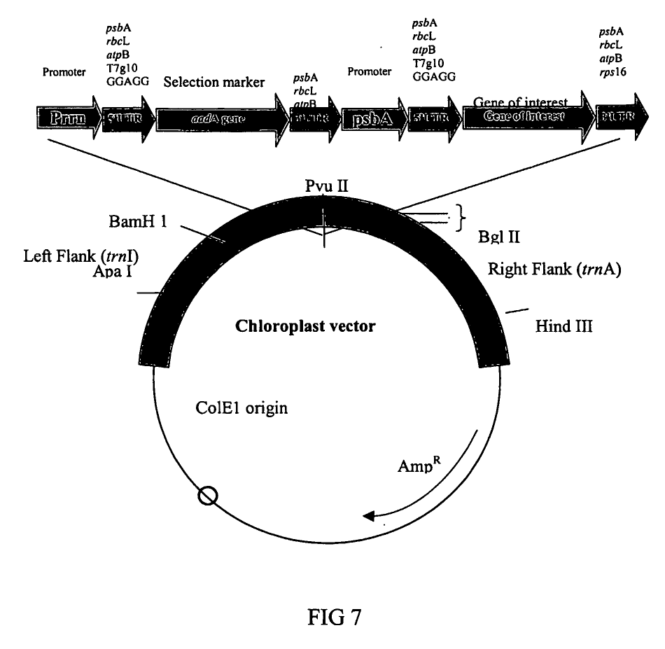

More specifically, FIG. 1. shows, integration of transgene cassettes into the chloroplast genome and study of homoplasmy. (a) Regions for homologous recombination are underlined in the native chloroplast genome. HSA is driven in all cassettes by the Prrn promoter upstream of the aadA gene for spectinomycin resistance with additional promoters and control elements as described in the text. Arrows within boxes show the direction of transcription. Numbers to the right indicate the predicted hybridizing fragments when total DNA digested with BamHI is probed with probe P1. (b) The 0.81 kb fragment (P1) flanking the cassette and 0.75 kb fragment containing HSA coding region (P2) were used as probes for the Southern blot analysis. (c, d) Southern blot analysis. 1: untransformed DNA; DNA from plants transformed with: 2,3: pLDAsdHSA; 4,5: pLDApsbAHSA. Plants for the first (T0) and second (T1) generation were analysed. 2,4: T0 generation. 3,5: T1 generation. Blots were probed with P1 (c) and P2 (d). AadA: aminoglycoside 3¢-adenylyl transferase; kb: kilobases; P: promoter; Prm: 16SrRNA promoter; SD: Shine-Dalgarno.

FIG. 2(a) shows transcription patterns of transgenic plants using a Northern Blot analysis. Lane 1 shows results from an untransformed plant; Lane 2 shows results for a plant transformed with pLDAsdHSA; Lane 3 shows a plant transformed with pLDApsbAHSA after illumination; and Lane 4 shows a plant transformed with pLDApsbAHSA and cultured in the dark.

FIG. 2(b) is a schematic view of the transcription patterns for the different cassettes integrated into the chloroplast genome.

More specifically, FIG. 2 shows, transcription patterns of transgenic plants. A Northern blot analysis was performed with total RNA extracted from leaves of potted plants. The 3′ of the psbA gene was used as probe. 1: untransformed plant; 2: transformed with pLDAsdHSA; 3: transformed with pLDApsbAHSA after illumination or 4: in the dark. Ethidium bromidestained rRNA was used to assess loading. Identified transcripts are indicated to the right. Horizontal arrows above genes show anticipated transcripts. Arrows within boxes show the orientation of genes within the chloroplast genome. Read through transcripts are not shown in this FIG.. rRNA: ribosomal RNA.

FIGS. 3(a-d) generally show analysis of HSA accumulation in transgenic chloroplasts.

FIG. 3(a) shows a graph illustrating an ELISA of HSA accumulation in leaves of potted plants at different stages of development. The stages include young, mature, and old. Samples were collected from untransformed plants or transformed with pLDAsdHSA or pLDApsbAHSA. Expression levels are indicated as a percentage of total protein.

FIG. 3(b) shows a graph illustrating an ELISA of HSA accumulation in leaves after different hours of illumination. Samples of leaves were collected from potted plants transformed with pLDApsbAHSA after the 8-hour dark period or at indicated hours in the light. The stages of the leaves include young, mature and old.

FIG. 3(c) shows a Coomassie stained gel designed to study HSA accumulation in tobacco leaves of potted plants. Total protein extracts were loaded in the gel. Lane 1 shows: 500 ng pure HSA; Lane 2 shows: molecular weight marker; Lane 3 shows: untransformed plant; Lane 4 shows: pLDAsdHSA; Lane 5 shows: pLDApsbAHSA after 8 hours of illumination; Lane 6 shows: pLDApsbAHSA after 8 hours of darkness. Between 40 and 50 mg of plant protein were loaded per well. The positions of HSA and RuBisCO large subunit (LSU) are marked.

FIG. 3(d) shows colorimetric immunoblot detection of tobacco protein extracts from mature leaves in potted plants. Total protein extracts were loaded in the gel. Lane 1 shows: 40 ng pure HSA; Lane 2shows: molecular weight marker; Lanes 3,5 show: untransformed plant extract; Lane 4 shows: pLDAsdHSA plant extract; Lane 6 shows: pLDApsbAHSA plant extract. Between 40 and 50 mg of plant protein were loaded per well.

FIGS. 4(a-d) generally show accumulation of HSA accumulation into inclusion bodies.

FIG. 4(a) shows Electron Micrographs of immunogold labeled tissues from untransformed plants.

FIGS. 4(b-d) show Electron Micrographs leaves transformed with the vector pLDApsbAHSA. Inclusion bodies are the small black circular dots.

FIG. 4(a-d) generally illustrates Study of HSA accumulation into inclusion bodies. (a-d) Electron micrographs of immunogold labelled tissues from untransformed (a) and transformed mature leaves with the chloroplast vector pLDApsbAHSA (b-d). Note presence of inclusion bodies (b-d) marked with an arrow in (d). Scale bars indicate mm. Magnifications are a ′ 10 000; b ′ 5000; c ′ 6300; d ′ 12 500.

FIG. 5 shows plant T1 phenotypes. Plants labeled 1 and 2 show untransformed plants. The plant labeled 3 shows a plant transformed with pLDAsdHSA. The plant labeled 4 shows plant transformed with pLDApsbAHSA.

FIGS. 6(a-b) show extraction from inclusion bodies.

FIG. 6(a) shows Silver stained SDS PAGE gel showing 1: 500 ng pure HSA; 2: molecular weight marker; soluble fraction obtained after centrifugation of pLDApsbAHSA transformed plant extract (lane 3) or untransformed plant extract (lane 4); 5: HSA after solubilization from the pellet; 6: proteins from untransformed plant, which followed the same process as the proteins of lane 5. Amounts of protein loaded per well were 10 mg in lanes 3 and 4, 550 ng in lane 5 and 450 ng in lane 6. HSA extraction from inclusion bodies. (a).

FIG. 6(b) shows the chemiluminiscent inmmunoblot detection of protein extracts. 1: 40 ng pure HSA; 2: HSA from a plant transformed with pLDApsbAHSA during the solubilization process, showing mono, di and trimeric forms; 3: proteins from an untransformed plant that followed the same process as the proteins for lane 2; 4: same HSA from lane 2 but in a more advanced stage of solubilization; 5: completely monomerized HSA after the end of the solubilization treatment (the sample of this lane corresponds with lane 5 in (a) ).

FIG. 7 shows a schematic view of generalized plastid vector.

### DETAILED DESCRIPTION OF THE INVENTION

In one preferred aspect, vectors are provided, which can be stably integrated into the plastid genome of plants for the variable-expression of Human Serum Albumin (HSA). In other preferred aspects methods of transforming plastid genomes to variable-express HSA, transformed plants and progeny thereof, which variable-express HSA are provided. Still another aspect provides for methods of variable-expressing biopharmaceutical proteins using selected regulatory elements. Another aspect provides for methods and constructs which protect biopharmaceutical proteins from proteolytic degradation.

The preferred aspects of this application are applicable to all plastids of higher plants. These plastids include the chromoplasts, which are present in the fruits, vegetables, and flowers; amyloplasts which are present in tubers such as potato; proplastids in the roots of higher plants; leucoplasts and etioplasts, both of which are present in the non-green parts of plants.

One aspect of this invention was to develop a more efficient expression system for human serum albumin, an important human therapeutic protein that is highly susceptible to degradation. Expression of HSA in mature plants under the translational control of SD sequence resulted in very low levels of HSA accumulation, probably due to excessive proteolytic degradation and poor rates of translation. However, when expressed under the control of psbA promoter and 5′UTR, up to a 500-fold increase in HSA accumulation was observed in mature plants compared to other regulatory sequences tested. HSA was observed to form large inclusion bodies, resulting even in a noticeable increase in the size of transgenic chloroplasts and presumably offering protection to HSA from proteolytic degradation. Inclusion bodies facilitated purification of HSA from other cellular proteins. The HSA molecule has a chemical and structural function rather than an enzymatic activity, therefore complex studies are necessary to fully demonstrate the functionality of the molecule (see Dodsworth et al., 1996; Ohtani et al., 1998; Petersen et al., 2000; Tarelli et al., 1998; Watanabe et al.

Definitions

To better understand the current disclosure, the following definitions, which shall hold their meaning throughout this application unless otherwise noted, are provided to put the application in proper context.

Variable-expression should be understood to mean the expression of HSA which yields variable amounts of HSA per gram of fresh weight of transgenic plants.

Properly folded should be understood to mean a protein that is folded into its normal conformational configuration, which is consistent with how the protein folds as a naturally occurring protein expressed in its native host cell.

Substantially homologous as used throughout the ensuing specification and claims, is meant a degree of homology to the native Human Serum Albumin sequence in excess of 60%, most preferably in excess of 80%, and even more preferably in excess of 90%, 95% or 99%. As was described in the application, the art has recognized a number of variants of the native HSA gene/protein. All of these variants are contemplated for use in this invention. For example the natural human serum albumin gene (JP-A-58-56684 corresponding to EP-A-73646, JP-A-58-90515 corresponding to EP-A-79739 and JP-A-58-150517 corresponding to EP-A-91527), has been well described in the art. However, one skilled in the art that there are modified human serum albumin genes (JP-A-62-29985 and JP-A-1-98486 corresponding to EP-A-206733), which have been identified and described. Substantial sequence identity or substantial homology as used herein, is used to indicate that a nucleotide sequence or an amino acid sequence exhibits substantial structural or functional equivalence with another nucleotide or amino acid sequence. Any structural or functional differences between sequences having substantial sequence identity or substantial homology will be de minimis; that is, they will not affect the ability of the sequence to function as indicated in the desired application. Differences may be due to inherent variations in codon usage among different species, for example. Structural differences are considered de minimis if there is a significant amount of sequence overlap or similarity between two or more different sequences or if the different sequences exhibit similar physical characteristics even if the sequences differ in length or structure. Such characteristics include, for example, ability to maintain expression and properly fold into the proteins conformational native state, hybridize under defined conditions, or demonstrate a well defined immunological cross-reactivity, similar biopharmaceutical activity, etc. Each of these characteristics can readily be determined by the skilled practitioner in the art using known methods.

In most cases, sequences having 95% homology to the sequences described herein will function as equivalents, and in many cases considerably less homology, for example 55% or 80%, will be acceptable. Locating the parts of these sequences that are not critical may be time consuming, but is routine and well within the skill in the art.

Spacer region is understood in the art to be the region between two genes. The chloroplast genome of plants contains spacer regions which highly conserved nuclear tide sequences. The highly conserved nature of the nuclear tide sequences of these spacer regions chloroplast genome makes the spacer region ideal for construction of vectors to transform chloroplast of a wide variety of plant species, without the necessity of constructing individual vectors for different plants or individual crop species. It is well understood in the art that the sequences flanking functional genes are well-known to be called “spacer regions”. The special features of the spacer region are clearly described in the Applicant's application Ser. No. 09/079,640 with a filing date of May 15, 1998 and entitled UNIVERSAL CHLOROPLAST INTEGRATION OF EXPRESSION VECTORS, TRANSFORMED PLANTS AND PRODUCTS THEREOF. The aforementioned application Ser. No. 09/079,640 is hereby incorporated by reference. It was well-known that there are at least sixty transcriptionally-active spacer regions within the higher plant chloroplast genomes (Sugita, M., Sugiura. M., Regulation of Gene Expression in Chloroplasts of Higher Plants, Plant Mol. Biol., 32: 315-326, 1996). Specifically, Sugita et al. reported sixty transcriptionally-active spacer regions referred to as transcription units, as can be seen in Table II of the article. Because the transcriptionally active spacer regions are known, a universal vector, as described in the Applicant's U.S. patent application Ser. No. 09/079,640, can be used in the identified spacer regions contained within a variety of the higher plant chloroplast genomes. By utilizing the teachings in Sugita et al., intergenic spacer regions are easily located in the plastid genome. Consequently this allows one skilled in the art to use the methods taught in the Applicant's U.S. patent application Ser. No. 09/079,640 to insert a universal vector containing the psbA, the 5′0 untranslated region (UTR) of psbA and the gene coding for HSA into the spacer regions identified by Sugita et al., and found across higher plants. The aforementioned applications and article are incorporated by reference.

Selectable marker provides a means of selecting the desired plant cells, vectors for plastid transformation typically contain a construct which provides for expression of a selectable marker gene. Marker genes are plant-expressible DNA sequences which express a polypeptide which resists a natural inhibition by, attenuates, or inactivates a selective substance, i.e., antibiotic, herbicide, or an aldehyde dehydrogenase such as Betaine aldehyde dehydrogenase (described in the Applicant's application Ser. No. 09/807,722 filed on Apr. 18, 2001, and herein fully incorporated by reference). Alternatively, a selectable marker gene may provide some other visibly reactive response, i.e., may cause a distinctive appearance or growth pattern relative to plants or plant cells not expressing the selectable marker gene in the presence of some substance, either as applied directly to the plant or plant cells or as present in the plant or plant cell growth media.

In either case, the plants or plant cells containing such selectable marker genes will have a distinctive phenotype for purposes of identification, i.e., they will be distinguishable from non-transformed cells. The characteristic phenotype allows the identification of cells, cell groups, tissues, organs, plant parts or whole plants containing the construct. Detection of the marker phenotype makes possible the selection of cells having a second gene to which the marker gene has been linked.

The use of such a marker for identification of plant cells containing a plastid construct has been described in the literature. In the examples provided below, a bacterial aadA gene is expressed as the marker. Expression of the aadA gene confers resistance to spectinomycin and streptomycin, and thus allows for the identification of plant cells expressing this marker. The aadA gene product allows for continued growth and greening of cells whose chloroplasts comprise the selectable marker gene product. Numerous additional promoter regions may also be used to drive expression of the selectable marker gene, including various plastid promoters and bacterial promoters which have been shown to function in plant plastids.

Inverted Repeat Regions are regions of homology, which are present in the inverted repeat regions of the plastid genome (known as IRA and IRB), two copies of the transgene are expected per transformed plastid. Where the regions of homology are present outside the inverted repeat regions of the plastid genome, one copy of the transgene is expected per transformed plastid.

Structural equivalent should be understood meaning a protein maintaining the conformational structure as the native protein expressed in its natural cell.

Vectors

The current application contemplates the use of vectors capable of plastid transformation, particularly of chloroplast transformation. Such vectors include chloroplast expression vectors such as pUC, pBR322, pBLUESCRIPT, pGEM, and all others identified by Daniel in U.S. Pat. No. 5,693,507 and U.S. Pat. No. 5,932,479. Included are also vectors whose flanking sequences are located outside of the embroidered repeat of the chloroplast genome. These publications and patents are hereby incorporated by reference to the same extent as if each individual publication or patent was specifically an individually indicated to be incorporated by reference.

The preferred aspect of this invention utilizes the universal integration and expression vector competent for stably transforming the plastid genome of different plant species (universal vector). The universal vector is described in WO 99/10513 which was published on Mar. 4, 1999, and application Ser. No. 09/079,640 which was filed on May 15, 1998, wherein both of said references are incorporated in their entirety.

As an illustrative aspect for the vectors, the Applicants created two chloroplast transformation vectors that were designed with different 5′ regulatory sequences to direct HSA expression and maximize protein accumulation in transgenic chloroplasts. Basic pLD vector, developed in this laboratory for chloroplast transformation, was used (Daniell et al., 1998; Daniell et al., 2001b; De Cosa et al., 2001; Guda et al., 2000; Kota et al., 1999). High levels of foreign protein expression in chloroplasts (3-21% of tsp) have been shown for different proteins using the SD 5′ sequence (Daniell et al., 2001b; DeGray et al., 2001; Kota et al., 1999).

In the plasmid pLDAsdHSA (FIG. 1b), the aadA gene, which confers spectinomycin resistance, and the HSA gene are expressed as a polycistron from the plastid Prrnpromoter. The Shine-Dalgarno (SD) consensus sequence GGAGG was placed upstream of both genes. In the pLDApsbAHSA vector (FIG. 1b), the 204 bp tobacco chloroplast DNA fragment containing the promoter and the psbA 5′UTR was inserted immediately upstream of the HSA coding sequence and downstream of the aadA gene. However it should be noted that the two vectors are illustrative examples and vectors can be constructed with different promoters as was described in U.S. patent application Ser. No. 09/079,640, different selectable markers such as those described in U.S. patent application Ser. No. 09/807,722, and different flanking sequences suitable for integration into a variety of plant plastid genomes.

General Methodology for Transforming the Plastid Genome

This illustrative example shows generally all of the necessary steps to practice the Applicants invention. Of course other suitable methods, which are known in the art may be substituted or used to supplement the example methodology described herein.

Isolation of Genomic DNA from Plants.

Mortar and pestle, liquid nitrogen, fresh dark green leaves. DNeasy Plant Mini Kit (QIAGEN Inc.)

PCR Amplification of Chloroplast Flanking Sequence.

Materials for PCR reaction: Genomic DNA (50-100 ng/μl), dNTPs, 10× pfu buffer, Forward primer, Reverse primer, autoclaved distilled H₂O and Turbo pfu DNA Polymerase.

Vector Construction.

1. Plasmid pUC19 or pBlueScript SK (+/−).

2. Species specific PCR amplified chloroplast DNA flanking sequences.

3. A promoter functional in plastids, 5′UTR of chloroplast gene, selectable marker gene, gene of interest and chloroplast 3′UTR.

4. Restriction enzymes and buffers.

5. T4 DNA polymerase to remove 3′ overhangs to form blunt ends and fill-in of 5′ overhangs to form blunt ends or Klenow large fragment (fill-in of 5′ overhangs to form blunt ends), alkaline phoshatase for dephoshorylation of cohesive ends, DNA ligase to form phosphodiester bonds and appropriate buffers.

6. Water baths or incubators set at different temperatures.

Preparation for Biolistics.

1. Autoclaved Whatman filter paper #1 (55 mm in diameter) dried in oven.

2. 100% ethanol.

3. Autoclaved tips in box, autoclaved kimwipes tissues wrapped in aluminum foil.

4. Sterile gold particles stored at −20° C. in 50% glycerol (see Notes 1 and 2).

5. Sterile rupture discs (1100 psi) and macrocarriers sterilized by dipping in 100% ethanol.

6. Autoclaved steel macrocarrier holders and stopping screens.

7. Freshly prepared 2.5 mM CaCl₂: weigh 1.84 g and dissolve in 5 mL H₂O and filter sterilized with 0.2 μm filter.

8. 0.1 M spermidine (highly hygroscopic): dilute 1M spermidine stock to 10× and aliquot 100 μL in 1.5 mL Eppendrop tubes to store at −20° C. Discard each tube after single use.

Medium Preparation for Plant Tissue Culture.

2.5.1. Tobacco.

Medium for 1000 mL: 4.3 g MS salts (INVITROGEN Inc.), H₂O (molecular biology grade), 100 mg/L myo-inositol, 1 mg/L thiamine-HCl, 3% sucrose for shoot induction and 2% sucrose for root induction, 1 mg/L 6-benzyl aminopurine (BAP; use 1 mL from 1 mg/mL stock), 0.1 mg/L indole-3- acetic acid (use 0.1 mL from 1 mg/mL IAA stock), 1 mg/L indole-3-butyric acid for root induction (use 1 mL from 1 mg/mL IBA stock). Add 500 mg/L spectinomycin in autoclaved medium when it cools to 45° C. −50° C. (use 5 mL filter sterilized spectinomycin from 100 mg/mL stock).

Edible Crops.

Potato.

Medium for 1000 mL: 4.3 g MS salts, B5 vitamins (make 100× solution in 100 mL H₂O by dissolving: 1 g myo-inositol, 10 mg nictonic acid, 10 mg pyridoxine-HCl, 100 mg thiamine-HCl; use 10 mL, store remaining solution at 4° C.), 5 mg/l zeatin riboside (use 0.5 mL from 1 mg/mL ZR stock), 0.1 mg/l α-napthaleneacetic acid (use 0.1 mL from 1 mg/mL NAA stock), 40 to 500 mg/L spectinomycin.

Tomato

Medium for 1000 mL: 4.3 g MS salts, B5 vitamins (10 mL from 10× stock), 0.2 mg/l indole-3-acetic acid (use 0.2 mL from 1 mg/mL IAA stock), 3 mg/l of 6-benzylaminopurine (use 3 mL from 1 mg/mL BAP stock). 300 or 500 mg/L spectinomycin.

For all plant growth media adjust to pH 5.8 with 1N KOH or 1N NaOH and add 6 g/L phytagel (Sigma) before autoclaving at 121° C. for 20 min. For preparation of 1 mg/mL stock of BAP, IAA, IBA, NAA, ZR respectively: weigh 10 mg powder and dissolve first in 1 or 2 drops of 1N NaOH and make up the final volume to 10 mL; store all plant growth regulators at 4° C. for 1-2 months).

Molecular Analysis of Transgenic Plants.

PCR Analysis for Gene Integration into Tobacco Chloroplasts

PCR reaction for 50 ,μL: 1.0 μl genomic DNA (50-100 ng/μl), 1.5 ,μl dNTPs (stock10 mM), 5.0 μl (10× PCR buffer), 1.5 μl Forward primer (to land on the native chloroplast genome; stock 10 μM), 1.5 μl Reverse primer (to land on the transgene; stock 10 μM), 39.0 μl autoclaved distilled H₂O and 0.5 μl Taq DNA polymerase.

Analysis of Homoplasmy by Southern Blots.

1. Depurination solution: 0.25 N HCl (use 0.4 mL HCl from 12.1 N HCl; Fisher Scientific USA, to make up final volume 500 mL with distilled H₂O).

2. Transfer buffer: 0.4 N NaOH, 1 M NaCl (weigh 16 g NaOH and 58.4 g NaCl and dissolve in distilled H₂O to make up the final volume to 1000 mL).

3. 20×SSC: 3M NaCl, 0.3 M sodium citrate trisodium salt (weigh 175.3 g NaCl, 88.2 g Na₃C₆H₅O₇.2H₂O 900 mL H₂O and adjust pH 7.0 using 1 N HCl and make up the final volume to 1000 mL with distilled H₂O and autoclave).

4. 2×SSC: Add 20 mL of 20×SSC in 180 mL of distilled H₂O.

Protein Analysis by Western Blots.

1. Acrylamide/Bis: ready made from Fischer (USA), stored at 4° C.

2. 10% SDS: dissolve 10 g SDS in 90 mL deionized water, make up the volume to 100 mL, store at room temperature.

3. Resolving gel buffer: 1.5 M Tris-HCl (add 27.23 g Tris base in 80 mL water, adjust to pH 8.8 with 6 N HCl and make up the final volume to 150 mL. Store at 4° C. after autoclaving).

4. Stacking gel buffer: 0.5 M Tris-HCl (add 6.0 g Tris base in 60 mL water. Adjust to pH 6.8 with 6 N HCl. Make up the volume to 100 mL. Store at 4° C. after autoclaving).

5. Sample Buffer (SDS Reducing Buffer): In 3.55 mL water add 1.25 mL 0.5 M Tris-HCl (pH 6.8), 2.5 mL glycerol, 2.0 mL (10% SDS), 0.2 mL (0.5% Bromophenol blue). Store at room temperature. Add 50 μL β-Mercaptoethanol (βME) to 950 μL sample buffer prior to its use.

6. 10× running buffer (pH 8.3): Dissolve 30.3 g Tris Base, 144.0 g Glycine and 10.0 g SDS in ˜700 mL water (add more water if not dissolving). Bring up the volume to 1 L and store at 4° C.

7. 10× PBS: Weigh 80 g NaCl, 2 g KCl, 26.8 g Na₂HPO₄.7 H₂O (or 14.4 g Na₂HPO₄), 2.4 g KH₂PO₄ in 800 mL water. Adjust pH to 7.4 with HCl and make up the volume to 1 L. Store at room temperature after autoclaving.

8. 20% APS: Dissolve 200 mg ammonium persulfate in 1 mL water (make fresh every two weeks).

9. Transfer buffer for 1500 mL: Add 300 mL 10× running buffer, 300 mL methanol, 0.15 g SDS in 900 mL water and make volume to 1 L.

Plant Extraction Buffer:

| | | Used Concentration | Final Concentration |
|---|---|---|---|
| 60 | μl | 5M NaCl | 100 mM |
| 60 | μl | 0.5 M EDTA | 10 mM |
| 600 | μl | 1 M Tris-HCl | 200 mM |
| 2 | μl | Tween-20 | .05% |
| 30 | μl | 10% SDS | 0.1% |
| 3 | μl | BME | 14 mM |
| 1.2 | mL | 1 M Sucrose | 400 mM |
| 1 | mL | Water | |
| 60 | μl | 100 mM PMSF | 2 mM |

Add PMSF just before use (vortex to dissolve PMSF crystals).

PMSF (Phenylmethyl sulfonyl fluoride): Dissolve 17.4 mg of powdered PMSF in 1 mL of methanol by vortexing and store at −20° C. for up to a month.

Methods

Isolation of Genomic DNA from Plants.

Extract the genomic DNA from fresh green leaves using DNeasy Plant kit (QIAGEN Inc.) following vender's instructions.

Amplification of Chloroplast Flanking Sequence.

Species-specific flanking sequences from the chloroplast DNA or genomic DNA of a particular plant species is amplified with the help of PCR using a set of primers that are designed using known and highly conserved sequence of the tobacco chloroplast genome.

Conditions for running PCR reaction: There are three major steps in a PCR, which are repeated for 30 to 40 cycles. (1) Denaturation at 94° C.: to separate double stranded chloroplast DNA. (2) Annealing at 54 to 64° C.: primers bind to single stranded DNA with formation of hydrogen bonds and the DNA polymerase starts copying the template. (3) Extension at 72° C.: DNA Polymerase at 72° C. extends to the template that strongly forms hydrogen bond with primers. Mismatched primers will not form strong hydrogen bonds and therefore, all these temperatures may vary based on DNA sequence homology. The bases complementary to the template are coupled to the primer on the 3′ side. The polymerase adds dNTPs from 5′ to 3′, reading the template in 3′ to 5′ direction and bases are added complementary to the template.

Chloroplast Transformation Vector.

The left and right flanks are the regions in the chloroplast genome that serve as homologous recombination sites for stable integration of transgenes. A strong promoter and the 5′ UTR and 3′ UTR are necessary for efficient transcription and translation of the transgenes within chloroplasts. For multiple gene expression, a single promoter may regulate the transcription of the operon, and individual ribosome binding sites must be engineered upstream of each coding sequence (2) (FIG. 10). The following steps are used in vector construction:

1. Amplification of flanking sequences of plastid with primers that are designed on the basis of known sequence of the tobacco chloroplast genome (between 16S-23S region of chloroplast).

2. Insert the PCR product containing the flanking sequence of the chloroplast genome into pUC19 plasmid digested with PvuII restriction enzyme (to eliminate the multiple cloning site), dephoshorylated with the help of alkaline phoshatase (CIP) for 5 min at 50° C. (to prevent recircularization of cloning vector). Inactivate CIP enzyme at 68° C. for 10 min.

Clone chloroplast transformation cassette (which is made blunt with the help of T4 DNA polymerase or Klenow filling) into a cloning vector digested at the unique PvuII site in the spacer region, which is conserved in all higher plants examined so far.

Delivery of Foreign Genes into Chloroplasts Via Particle Gun.

This is most successful and a simple technique to deliver transgenes into plastids and is referred as Biolistic PDS-1000/ He Particle Delivery System (18,19). This technique has proven to be successful for delivery of foreign DNA to target tissues in a wide variety of plant species and integration of transgenes has been achieved in chloroplast genomes of tobacco (2), Arabidopsis (20), potato (21), tomato (25) and transient expression in wheat (22), carrot, marigold and red pepper (23) (see Note 5).

Preparation of Gold Particle Suspension.

1. Suspend 50-60 mg gold particles in 1 mL 100% ethanol and vortex for 2 min.

2. Spin at maximum speed ˜10,000×g (using tabletop microcentrifuge) for 3 min.

3. Discard the supernatant.

4. Add 1 ml fresh 70% ethanol and vortex for 1 min.

5. Incubate at room temperature for 15 min and shake intermittently.

6. Spin at 10,000×g for 2 min.

7. Discard supernatant, add 1 ml sterile distilled H₂O, vortex for 1 min, leave at room temperature for 1 min, and spin at 10,000×g for 2 min.

8. Repeat above washing process three times with H₂O (step 7).

9. Resuspend the gold-pellet in 1 mL 50% glycerol, store stock in −20° C. freezer.

Precipitation of the Chloroplast Vector on Gold Particles for Five Samples.

1. Take 50 μl the gold particles in 1.5 mL tube after vortexing for a 1 min.

2. Add 10 μl DNA (about 1 μg/μl plasmid DNA), and vortex the mixture for 30 sec.

3. Add 50 μl of 2.5 M CaCl₂ and vortex the mixture for 30 sec.

4. Add 20 μl of 0.1 M spermidine and vortex the mixture for 20 min at 4° C.

Washing of Chloroplast Vector Coated on Gold Particles.

1. Add 200 μl 100% ethanol and vortex for 30 sec.

2. Spin at 3000×g for 40 sec.

3. Pour off ethanol supernatant.

4. Repeat ethanol washings five times.

5. In the last step, pour off ethanol carefully and add 35-40 μl ethanol (100%).

Preparation of Macrocarriers.

1. Sterilize macrocarriers by dipping in 100% ethanol for 15 min and insert them into sterile steel ring holder with the help of a plastic cap when air-dried.

2. Vortex the gold-plasmid DNA suspension and pipet 8-10 μl in the center of macrocarrier and let it air dry.

Gene Gun Setup for Bombardment of Samples.

1. Wipe the gun chamber and holders with 100% ethanol using fine tissue paper (do not wipe the door with alcohol).

2. Turn on the vacuum pump.

3. Turn on the valve (Helium pressure regulator) of Helium gas tank (anti-clockwise).

4. Adjust the gauge valve (adjustable valve) ˜200 to 250 psi above the desired rupture disk pressure (clockwise) using adjustment handle.

5. Turn on the gene gun.

6. Place the rupture disc (sterilized by dipping in 100% ethanol for 5 min) in the rupture disc-retaining cap and tightly screw to the gas acceleration tube.

7. Place a stopping screen in the macrocarrier launch assembly and above that place macrocarrier with gold particles with chloroplast vector facing down towards screen. Screw assembly with a macrocarrier coverlid and insert in the gun chamber.

8. Place an intact leaf or explants to be bombarded on a filter paper (Whatman No. 1) soaked in medium containing no antibiotics. Place sample plate over target plate shelf, insert in the gun chamber and close the bombardment chamber door.

9. Press Vac switch to build pressure (up to 28 inches of Hg) in the vacuum gauge display. Turn same switch down at hold point and press Fire switch until you hear a burst sound of the ruptured disc.

10. Press Vent switch to release the vacuum and open the chamber to remove sample.

11. Shut down the system by closing the main valve (Helium pressure regulator) on the Helium gas cylinder. Create some vacuum in the gene gun chamber and keep using fire switch on and off until both pressure gauges' show zero reading. Release the vacuum pressure and turn off the gene gun and vacuum pump.

12. Incubate bombarded sample plates in the culture room for two days in the dark (i.e. covered with aluminum foil) and on the third day cut explants in appropriate pieces and place on the selection medium.

Plant Tissue Culture and Chloroplast Transformation.

Tobacco Chloroplast Transformation.

A highly efficient and reproducible protocol has been established for Nicotiana tabacum cv. Petit Havana (Daniell, H. (1997) Methods in Mol. Biol. Recombinant gene expression protocols. 62,463-489.

1. Bombard 4 weeks old dark green tobacco leaves on the abaxial (bottom side) side with the chloroplast vector and incubate leaves in the dark for 2 days on selection free medium.

2. On the third day cut bombarded leaf explants into small square pieces (5 mm) and place explants facing abaxial surface towards selection medium containing MS salts, 1 mg/l thiamine HCl, 100 mg/l myo-inositol, 3% sucrose, 1 mg/l BAP and 0.1 mg/I IAA along with 500 mg/l spectinomycin as a selective agent.

3. Transgenic shoots should appear after three to five weeks of transformation. Cut the shoot leaves again into small square explants (2 mm) and subject to a second round of selection for achieving homoplasmy on fresh medium.

4. Regenerate transgenic shoots (confirmed by PCR for transgene integration) on rooting medium containing MS salts, 1 mg/l thiamine HCl, 100 mg/l myo-inositol, 2% sucrose and 1 mg/l IBA with 500 mg/l spectinomycin.

5. Transfer transgenic plants into pots under high humidity and move them to green house or growth chamber for further growth and characterization.

Plastid Transformation of Edible Crops.

The concept of universal vector for using the chloroplast DNA from one plant species to transform another species (of unknown sequence) was developed by the Daniell group (8). Using this concept both tomato and potato chloroplast genomes were transformed as described below.

Potato Chloroplast Transformation.

Using the tobacco chloroplast vector, leaf tissues of potato cultivar FL1607 was transformed via biolistics, and stable transgenic plants were recovered using the selective aadA gene marker and the visual green fluorescent protein (GFP) reporter gene (21).

1. Bombard potato leaves (3-4 week old) and incubate in the dark for 2 days on selection free medium.

2. Third day excise leaves into small square pieces (5 mm) and place on MS medium containing B5 vitamins, 5 mg/l ZR, 0.1 NAA, and 3% sucrose. Gradually increase spectinomycin selection pressure (40 to 400 mg/l) after every two weeks subculture under diffuse light.

3. Regenerate shoots from transgenic potato calli on MS medium containing B5 vitamins, 0.01 mg/L NAA, 0.1 mg/L GA3, 2% sucrose and 40-400 mg/l spectinomycin.

4. Transfer transgenic shoots on basal MS medium containing B5 vitamins, 2% sucrose and 40-400 mg/l spectinomycin for root induction. Transfer transgenic plantlets to growth chamber.

Tomato Chloroplast Transformation.

Using the tobacco chloroplast vector, tomato (Lycopersicon esculentum cv. IAC Santa Clara) plants with transgenic plastids were generated using very low intensity of light (25).

1. Bombard four-week-old tomato leaves and incubate in the dark for 2 days on selection free medium.

2. Excise bombarded leaves into small pieces and place on shoot induction medium containing 0.2 mg/L IAA, 3 mg/L BAP, 3% sucrose and 300 mg/L spectinomycin.

3. Select spectinomycin-resistant primary calli after a three to four month duration without any shoot induction.

4. Regenerate shoots in about four weeks after transfer of transgenic calli to shoot induction medium containing 0.2 mg/L IAA, 2 mg/L ZR, 2% sucrose and 300 mg/L spectinomycin then root on hormone-free medium. Transfer regenerated transgenic plants into the greenhouse.

Molecular Analysis of Transgenic Plants.

PCR Screening of Transgenic Shoots.

This method has been used to distinguish between mutants, nuclear and chloroplast transgenic plants. By landing one primer on the native chloroplast genome adjacent to the point of integration and a second primer on the aadA gene (26. PCR product of an appropriate size should be generated in chloroplast transformants. Since this PCR product cannot be obtained in nuclear transgenic plants or mutants, the possibility of nuclear integration or mutants should be eliminated.

1) Extract the genomic DNA from transgenic leaf tissue using DNeasy Plant kit (QIAGEN Inc.) by following vender's instructions. For lower amount of transgenic tissues, volume of buffers may be reduced appropriately.

2) Run PCR reaction with Taq DNA Polymerase (QIAGEN Inc.) using appropriate primers following the same conditions as described above for amplification of flanking sequences.

Analysis of Homoplasmy by Southern Blot.

In Southern blot analysis, tobacco plastid genome digested with suitable restriction enzymes should produce a smaller fragment (flanking region only) in wild type plants compared to transgenic chloroplast that include transgene cassette as well as the flanking region. In addition, homoplasmy in transgenic plants is achieved when only the transgenic fragment is observed.

Transfer of DNA to Membrane.

1. Digest the genomic DNA (˜2 to 10 μg) with suitable restriction enzymes from transgenic samples (including wild type as a control) and run digested DNA on 0.8% agarose gel containing 5 μL EtBr (from 10 mg/mL stock) in 100 mL for four hours at 40V.

2. Soak gel in 0.25 N HCl (depurination solution) for 15 minutes and rinse gel twice in distilled H₂O for 5 minutes.

3. Soak gel for 20 minutes in transfer buffer to denature DNA.

4. Transfer overnight DNA from gel to nylon membrane (pre-soak first in water, then in transfer buffer for 5 minutes) using the transfer buffer.

5. Next day, rinse membrane twice with 2×SSC buffer for 5 minutes each and air-dry for 5 minutes on filter papers. Cross-link transferred DNA to membrane using GS GeneLinker UV Chamber (Bio-Rad) at appropriate (C3) setting.

Preparation of Probe.

1. Digest any plasmid (containing flanking sequences of the chloroplast genome) with appropriate restriction enzymes.

2. Denature 45 μL flanking DNA fragment (50-250 ng) at 95° C. for 5 minutes, then place on ice for 2-3 minutes.

3. Add denatured probe to Ready-To-Go DNA Labeling Beads (-dCTP) tube (Amersham Biosciences, USA) and gently mix by flicking the tube.

4. Add 5 μL radioactive α³²P (dCTP; Amersham Biosciences, USA) to probe mixture and incubate at 37° C. for 1 hour and filter the probe using ProbeQuant G-50 Micro Columns (Amersham Pharmacia Biotech Inc. USA).

Prehybridization and Hybridization.

Place the blot (DNA transfer side facing towards the solution) in a hybridization bottle and add 10 mL Quik-Hyb (Stratagene, USA).

Incubate for 1 hour at 68° C. Add 100 μL sonicated salmon sperm (10 mg/mL stock; Stratagene, USA) to the labeled probe and heat at 94° C. for 5 minutes and add to bottle containing membrane and Quik-Hyb solution. Incubate for 1 hour at 68° C.

Washing and Autoradiography.

1. Discard Quik-Hyb solution with probe and wash membrane twice in 50 mL (2×SSC buffer and 0.1% SDS) for 15 minutes at room temperature.

2. Wash membrane twice in 50 mL (0.1×SSC buffer and 0.1% SDS) for 15 minutes at 60° C.

3. Wrap the wash membrane in saran wrap and expose blot to x-ray film in the dark and leave at −70° C. until ready for development.

Determination of Transgene Expression by Western Blot.

Extraction of Plant Protein.

1. Grind 100 mg of leaf in liquid nitrogen and add 200 μL of extraction buffer to samples on ice.

2. Add appropriate volume of freshly prepared 2× Sample loading buffer to an aliquot plant extract (from a stock containing 50 μL β-mercaptoethanol and 950 μL sample loading buffer).

3. Boil samples for 4 minutes with loading dye and centrifuge for 2 minutes at 10,000×g, then immediately load 20 μL samples into gel.

Running Gel.

Load samples on gel and run for half hour at 100 V, then I hour at 150 V until the marker bands corresponding to your protein are in middle.

Transfer of Protein to Membrane.

Transfer protein from gel to membrane using Mini Transfer Blot Module at 30 V overnight or 65 V for 2 hours or 100 V for 1 hour. Membrane wrapped in saran wrap can be stored at −20° C. for a few days if necessary.

Membrane Blocking

1. After transfer, rinse membrane with water and incubate membrane in PTM (100 mL 1×PBS, 50 μL 0.05% Tween 20, and 3 g dry milk (3%/o) for 1 hour at room temperature.

2. Add primary antibody in suitable dilution for 15 mL and incubate for 2 hours at room temperature. Wash membrane twice with 1×PBS for 5 minutes each.

3. Add secondary antibody in proper dilution for 20 mL. Incubate for 1.5 hours at room temperature on a shaker.

4. Wash twice with PT (100 ml 1×PBS+50 μL Tween 20) for 15 minutes and finally with 1×PBS for 10 minutes.

Exposure of the Blot to X-ray Film.

1. Mix 750 μL of each chemiluminescent solution (Luminol Enhancer and Stable Peroxide) in 1.5 mL tube and add to membrane, cover thoroughly.

2. Wipe out extra solution and expose blot to x-ray film for appropriate duration and develop film.

Seed Sterilization.

1. Vortex small amount of seeds into microcentrifuge tube with 1 mL 70% ethanol for 1 minute. Discard ethanol after brief spin.

2. Add 1 mL disinfecting solution (1.5% Bleach and 0.1% Tween 20) in tube and vortex intermittently for 15 min. Discard solution after brief spin.

3. Wash the seed thrice with sterile distilled water.

4. Spray seeds with sterile water on plate containing RMOP basal medium supplemented with 500 μg/mL spectinomycin to determine maternal inheritance in transgenic chloroplast plants.

Evaluation of Results.

Maternal Inheritance in Chloroplast Transgenic Plants.

Transgenes integrated into chloroplast genomes are inherited maternally. This is evident when transgenic seed of tobacco are germinated on RMOP basal medium containing 500 μg/mL spectinomycin. There should be no detrimental effect of the selection agent in transgenic seedlings whereas untransformed seedlings will be affected.

CTB-GMI-Gangliosides Binding ELISA Assay.

1. Coat microtiter plate (96 well ELISA plate) with monosialoganglioside-GM1 {3.0 μg/mL in bicarbonate buffer (15 mM Na₂CO₃, 35 mM NaHCO₃, pH 9.6)} and as a control, coat BSA (3.0 ug/mL in bicarbonate buffer) in few wells.

2. Incubate plate overnight at 4° C.

3. Block wells with 1% (w/v) bovine serum albumin (BSA) in 0.01 M phosphate-buffered saline (PBS) for two hours at 37° C.

4. Wash wells thrice with PBST buffer (PBS containing 0.05% Tween 20).

5. Incubate plate by adding soluble protein from transformed and untransformed plants and bacterial CTB in PBS.

6. Add primary antibodies (rabbit anti cholera serum diluted 1:8000 in 0.01 M PBST containing 0.5% BSA) and incubate plate for 2 hours at 37° C.

7. Wash well thrice with PBST buffer.

8. Add secondary antibodies diluted 1:50,000 (mouse anti rabbit IgG-alkaline phosphatase conjugate in 0.01 M PBST containing 0.5% BSA) and incubate plate for 2 hours at 37° C.

9. Develop plate with Sigma Fast pNPP substrate. Stop reaction by adding 3 M NaOH and read plate absorbance at 405 nm.

The macrophage lysis assay is as follows:

1. Isolate crude extract protein from 100 mg transgenic leaf using 200 μL of extraction buffer containing CHAPS detergent (4% CHAPS, 10 mM EDTA, 100 mM NaCl, 200 mM Tris-HCl, pH 8.0, 400 mM sucrose, 14 mM β-mercaptoethanol, 2 mM PMSF) and one without CHAPS detergent.

2. Spin samples for five minutes at 10,000×g and use both supernatant and homogenate for assay

3. Plate macrophage cells RAW 264.7 (grown to 50% confluence) into 96-wells plate, incubated in 120 μL Dulbecco's Modified Eagle's Medium (DMEM; from Invitrogen life technologies).

4. Aspirate medium from wells and add 100 μL medium containing 250 ng/mL proteins in crude leaf extract.

5. In control plate, add only DMEM with no leaf fraction to test toxicity of plant material and buffers.

6. In another plate, add 40 μL dilutions onto RAW 264.7 cells from plant samples, which serially diluted 2 fold, so that the top row had plant extract at 1:14 dilution.

7. Add 20 μL of MTT 3-[4,5-dimethylthiazol-2-yl]-2,5-diphenyltetrazolium bromide; Sigma) to each well containing cells (from a stock 5 mg/ml MTT dissolved in 1×PBS and filter sterilize) after 5 hours to assess the cell death.

8. Incubate the plate at 37° C. for 5 hours. Remove media with needle and syringe. Add 200 μL of DMSO to each well and pipette up and down to dissolve crystals. Transfer to plate reader and measure absorbance at 550 nm.

Active PA was found in both the supernatant and homogenate fractions. However, maximum macrophage lysis activity was noticed in supernatant when extraction buffer was used with CHAPS detergent.

Cholera Toxin (CTB) Antigen as an Edible Vaccine.

Chloroplast transgenic plants are ideal for production of vaccines. The heatlabile toxin B subunits of E. coli enterotoxin (LTB), or cholera toxin of Vibrio cholerae (CTB) have been considered as potential candidates for vaccine antigens. Integration of the unmodified native CTB gene into the chloroplast genome has demonstrated high levels of CTB accumulation in transgenic chloroplasts (Daniell, H., et al. (2001). J. MoL Biol. 311,1001-1009.). This new approach not only allowed the high level expression of native CTB gene but also enabled the multimeric proteins to be assembled properly in the chloroplast, which is essential because of the critical role of quaternary structure for the function of many vaccine antigens. The expression level of CTB in transgenic plants was between 3.5% and 4.1% tsp and the functionality of the protein was demonstrated by binding aggregates of assembled pentamers in plant extracts similar to purified bacterial antigen, and binding assays confirmed that both chloroplast-synthesized and bacterial CTB bind to the intestinal membrane GM1-ganglioside receptor, confirming correct folding and disulfide bond formation of CTB pentamers within transgenic chloroplasts (FIG. 11).

Oral Delivery of Vaccines and Selection of Transgenic Plants without the Use of Antibiotic Selectable Markers.

Betaine aldehyde dehydrogenase (BADH) gene from spinach has been used as a selectable marker to transform the chloroplast genome of tobacco (Daniell, H. et al., (2001) Curr. Genet. 39,109-116). Transgenic plants were selected on media containing betaine aldehyde (BA). Transgenic chloroplasts carrying BADH activity convert toxic BA to the beneficial glycine betaine (GB). Tobacco leaves bombarded with a construct containing both aadA and BADH genes showed very dramatic differences in the efficiency of shoot regeneration. Transformation and regeneration was 25% more efficient with BA selection, and plant propagation was more rapid on BA in comparison to spectinomycin. Chloroplast transgenic plants showed 15 to 18 fold higher BADH activity at different developmental stages than untransforned controls. Expression of high BADH level and resultant accumulation of glycine betaine did not result in any pleiotropic effects and transgenic plants were morphologically normal and set seeds as untransformed control plants.

Production of Human Therapeutic Proteins in Transgenic Chloroplasts.

Human Serum Albumin (HSA) Protein,

Human Serum Albumin (HSA) accounts for 60% of the total protein in blood and widely used in a number of human therapies. Chloroplast transgenic plants were generated expressing HSA (Fernandez-San Millan et al., (2003) Plant Bitechnol. J. 1,71-79). Levels of HSA expression in chloroplast transgenic plants was achieved up to 11.1% tsp. Formation of HSA inclusion bodies within transgenic chloroplasts was advantageous for purification of protein. Inclusion bodies were precipitated by centrifugation and separated easily from the majority of cellular proteins present in the soluble fraction with a single centrifugation step. Purification of inclusion bodies by centrifugation may eliminate the need for expensive affinity columns or chromatographic techniques.

Purification of HSA.

1. Solubilize the HSA inclusion bodies from transformed tissues using extraction buffer containing 0.2M NaCl, 25 mM Tris-HCl (pH 7.4), 2 mM PMSF and 0.1 % Triton X-100.

2. Spin at 10,000×g. Suspend the pellet in buffer containing 6M Gu-HCl, 0.1M βME and 0.25 mM Tris-HCl (pH 7.4).

3. Dilute plant extract 100-fold in buffer containing 100 mM NaCl, 50 mM Tris-HCl (pH 8.5) and 1 mM EDTA.

4. Concentrate HSA protein by precipitation using a polyethylenglycol treatment at 37%.

5. Separate protein fractions by running a SDS-PAGE gel and stain gel with silver regent following vender's instruction (Bio-Rad, USA).

Electron Microscopy and Immunogold Labeling.

1. Cut the transformed and untransformed leaf in 1-3 mm squares.

2. Fix them in 0.1 M cacodylate buffer pH 7.4 (2.5% glutaraldehyde, 2% paraformaldehyde and 5 mM CaCl₂) for 15 minutes under vacuum and 12 hours at 4° C.

3. Rinse samples twice in 0.1M cacodylate buffer (pH 7.4) after fixation.

4. Dehydrate fixed samples through a graded ethanol series to 95%, then implant in LRW resin at 60° C. for 24 hours.

5. Cut ultra-thin sections using a Leica Ultracut T ultramicrotome and collect sections onto nickel grids.

6. Incubate sections in 0.05M glycine prepared in PBS buffer for 15 minutes to inactivate residual aldehyde groups.

7. Place grids onto drops of blocking solution (PBS containing 2% non-fat dry milk) and incubate for 30 minutes

8. Incubate sections for 1 hour in a goat anti-human albumin polyclonal antibody (dilution range from 1:1000 to 1:10,000 in blocking solution).

9. Wash sections with blocking solution 6×5 minutes each.

10. Incubate sections for 2 hours with a rabbit anti-goat IgG secondary antibody conjugate to 10 nm gold diluted 1:40 in blocking solution.

11. Wash sections 6×5 minutes in blocking solution and 3×5 minutes with PBS, and fixed sections in 2% glutaraldehyde diluted in PBS for 5 minutes.

12. Wash fixed sections in PBS 3×5 minutes, then in distilled water 5×2 min each.

13. Stain sections using uranyl acetate and lead citrate and examine samples under transmission electron microscope at 60kv.

Notes

1. Gold particles suspended in 50% glycerol may be stored for several months at −20° C. Avoid refreezing and thawing spermidine stock; use once after thawing and discard the remaining solution. Use freshly prepared CaCl₂ solution after filter sterilization. Do not autoclave.

2. Precipitation efficiency of DNA on gold and spreading of DNA-gold particles mixture on macrocarriers is very important. For high transformation efficiency via biolistics, a thick film of gold particles should appear on macrocarrier disks after alcohol evaporation. Scattered or poor gold precipitation reduces the transformation efficiency.

3. Generally, a 1000 bp flanking sequence region on each side of the expression cassette is adequate to facilitate stable integration of transgenes.

4. Use of the 5′ untranslated region (5′ UTR) and the 3′ untranslated region (3′ UTR) regulatory signals are necessary for higher levels of transgene expression in plastids (13). The expression of transgene in the plant chloroplast depends on a functional promoter, stable mRNA, efficient ribosomal binding sites; efficient translation is determined by the 5′ and 3′ untranslated regions (UTR). Chloroplast transformation elements Prrn, psbA5′UTR, 3′UTR can be amplified from tobacco chloroplast genome.

5. Bombarded leaves after two-days dark incubation should be excised in small square pieces (5-7 mm) for first round of selection and regenerated transgenic shoots should be excised into small square pieces (2-4 mm) for a second round of selection.

6. Temperature for plant growth chamber should be around 26-28° C. for appropriate growth of tobacco, potato and tomato tissue culture. Initial transgenic shoot induction in potato and tomato require diffuse light. However, higher intensity is not harmful for tobacco.

7. Transformation efficiency is very poor for both potato and tomato cultivars compared to tobacco.

8. Tobacco chloroplast vector gives low frequency of transformation if used for other plant species. For example, when petunia chloroplast flanking sequences were used to transform the tobacco chloroplast genome (DeGray, G. et al., (2001), Plant Physiol. 127,852-862.), it resulted in very low transformation efficiency.

Under diffuse light conditions, highly regenerating tomato cultivar (Microtom) shoots produce premature flowering that inhibit further growth of transgenic plants. Therefore, after the first shoot induction phase, shoots should be moved to normal light conditions.

### ILLUSTRATIVE EXAMPLE

Reference will now be made in detail to the presently preferred aspects of the invention, which, together with the following example, serve to explain the principals of the invention. The following example is intended as a non-limiting example of the Applicants, and is no way to a intended as a limitation

As was earlier described, two chloroplast transformation vectors, the pLDApsbAHSA vector, and the pLDAsdHSA vector, were designed with different 5′ regulatory sequences to direct HSA expression and maximize (variable-express)HSA protein accumulation in transgenic chloroplast. The prior art has consistently found that foreign genes under the control of the psbA promoter and untranslated region of psbA are expressed at very high levels (Daniell et al., 1990). This enhancement of translation may be due to elements in the 5′UTR (Eibl et al., 1999).

Vector integration

Vectors were bombarded into tobacco leaves as described previously (Daniell, 1997) and, after 5 weeks, several primary shoots appeared from each bombarded leaf as a result of independent transformation events. Putative transformed shoots were identified by growth on 500 mg/mL of spectinomycin.

Integration of the foreign gene cassettes into the chloroplast genome was confirmed by PCR screening of primary shoots. The strategy employed lands one primer on the native chloroplast genome adjacent to the point of integration and the second primer on the aadA gene. This PCR product can not be obtained in nuclear transgenic plants or spontaneous mutants, thus both possibilities could be eliminated. It was found that 90% of total shoots obtained were true chloroplast transformants. Confirmed transformants were subjected to a second round of spectinomycin selection to achieve homoplasmy. They were rooted in the presence of spectinomycin and then transferred to pots for further characterization. Southern blot analysis was performed, as can be seen in FIG. 1(a), to select homoplasmic T0 lines and confirm stable maintenance of integrated transgenes in the T1 generation (FIG. 1b-d). The flanking region probe (P1) identified a 0.45 kb fragment in the untransformed control plant, as expected (FIG. 1c). In the chloroplast transgenic lines, only transformed genome copies are observed as evidenced by the 10.5 and 10.7 kb hybridizing fragments for pLDAsdHSA and pLDApsbAHSA transgenic lines, respectively. To confirm that the 10.5 and 10.7 kb fragments contained the HSA gene, the same blot was reprobed with the HSA P2 probe. As expected, hybridization was detected only in the chloroplast transgenic lines (FIG. 1d). Absence of other hybridizing fragments eliminates nuclear and chloroplast integration events in the same transgenic line. HSA quantities in transgenic tobacco chloroplasts were determined by ELISA. More than a 360-fold difference in HSA accumulation was observed between plants transformed with the two different vectors (FIG. 3a): 0.02% vs. 7.2% tp in DLDAsdHSA and pLDApsbAHSA transgenic lines, respectively.

Chloroplast constructs with the SD sequence have been demonstrated to direct CTB expression very efficiently (up to 4% of tsp; Daniell et al., 2001b). Similar constructs, but inserted in other areas of the plastid genome, have also been successful (3-21% tsp; DeGray et al., 2001; Kota et al., 1999), demonstrating that high protein expression levels can be achieved by using this construct in an operon with a SD sequence. Thus, low levels of HSA expression in the pLDAsdHSA transgenic plants could not be due to the effect of regulatory signals in the construct. Differences in the amounts of HSA are most likely due to post-transcriptional, translational or post-translational effects. To study differences in HSA expression, transcript abundance was examined by Northern blots, which were performed using the 3′ psbA region as the probe (FIG. 2). The 5′ psbA/HSA monocistron transcript is much more abundant than the aadA/SD/HSA dicistron, but such differences do not show a linear correlation with the 360-fold difference in HSA accumulation between both transgenic lines. Such a lack of correlation between transcript abundance and protein accumulation has been reported from several laboratories when the psbA 5′UTR is used (Mayfield et al., 1995, suggesting an important role of the psbA 5′UTR in enhancement of translation. Eibl et al. (1999) also showed that deletion of the terminal sequences of the psbA 5′ UTR decreased the ability of the UTR to enhance translation. Thus, efficient translation of the 5′ UTR in the pLDApsbAHSA transgenic line might be an important factor in establishing high levels of HSA accumulation.

There are several studies demonstrating that psbA 5′UTR confers light-dependent translation not only to the psbA gene (Zerges, 2000) but also to other heterologous proteins (Eibl et al., 1999). Expression of HSA under the psbA 5′UTR control is light dependent. Changes in HSA accumulation after different periods of illumination were monitored by ELISA(FIG. 3b). HSA quantity was observed to be maximum up to 50 hours of continuous illumination (1 1.1% of tp) in mature leaves and a 2-4-fold decrease was observed after the 8 hours dark period. Such differences in HSA accumulation were so pronounced that it was detected by staining gels with Coomassie Brilliant Blue (FIG. 3c).Eibl et al. (1999) showed that although translation is arrested in the dark, the 5′ psbA/uidA MRNA turnover was very low.

This observation was confirmed for HSA by Northern blot analysis, which showed no major differences between light and dark amount of 5′ psbA/HSA transcripts (FIG. 2, lanes 3, 4). Therefore, differences in HSA accumulation between dark and light could not be due to differences in the rate of transcription or transcript stability, but due to the arrest of translation in the dark and the turnover of HSA in the chloroplast. Proteins from transformed plants were separated to study the pattern of HSA accumulation within transgenic chloroplasts. Western blots confirmed differences in HSA quantities among transgenic lines (FIG. 3d). In pLDApsbAHSA transgenic lines, HSA is partially solubilized with the standard buffer used for total protein extraction. This observation suggests formation of HSA aggregates inside transgenic chloroplasts in the pLDApsbAHSA transformants. Electron microscopy and immunogold labeling therefore were performed in transformed and untransformed plants to further investigate this. As expected, electron micrographs of leaf tissues showed formation of large aggregates or inclusion bodies within transgenic chloroplasts of pLDApsbAHSA mature transformed plants (FIG. 4b-d). Chloroplasts containing inclusion bodies increased in size to accommodate large accumulation of HSA (compare FIGS. 4a,d). However, the phenotype of these plants remained normal (FIG. 5). The amount of HSA in pLDAsdHSA transgenic chloroplasts was so low that it was not possible to detect immunogold labeling above the background. No significant changes in chloroplast size were observed in these plants. Inclusion bodies have been often observed in the cytosol of prokaryotes and eukaryotes when heterologous proteins are over-expressed.

The occurrence of this feature in the chloroplast was first reported by Ketchner et al. (1995). It is widely known that protein aggregation into inclusion bodies mostly involves intermolecular associations of partially folded intermediates (Mitraki and King, 1989). High protein concentrations usually lead to conditions that frequently exceed the normal solubility limit. Even the most abundant protein in photosynthetic cells, RuBisCO, forms inclusion bodies in some cases. Many autotrophic bacteria and all cyanobacteria package much of the RuBisCO into inclusion bodies actively involved in the fixation of CO₂, known as carboxysomes (Shively and English, 1991). This application has shown, based on the process of formation of inclusion bodies, that in contrast to pLDAsdHSA transgenic lines, HSA synthesized under the psbA5′UTR forms large aggregates mainly due to the high local concentration of the protein. Formation of inclusion bodies is one of the strategies for reducing the proteolysis of unstable recombinant proteins (Enfors, 1992). The majority of recombinant proteins studied have been shown to be highly resistant to proteolysis inside inclusion bodies. Although there is protection from proteases within inclusion bodies, some proteolysis can also take place directly on the aggregated protein (Carrio et al., 1999). Our results indicated HSA also appears to be susceptible to some proteolytic degradation within transgenic chloroplasts. However, the net balance between synthesis and degradation is highly favorable, especially after several hours of continuous illumination.

Properly folded HSA can be recovered from inclusion bodies after denaturation for complete solubilization and in vitro refolding. Proper refolding of HSA from inclusion bodies is a routine procedure that has been previously demonstrated in several studies with E. coli (Latta et al. (1987) and Saccharomyces cerevisiae (Dodsworth et al., 1996; Quirk et al., 1989). In these cases, human and recombinant refolded HSA were compared and it was shown that the two proteins were structurally equivalent, demonstrating that HSA may be recovered from inclusion bodies and properly folded to maintain human therapeutic value.

Following the guidelines from these protocols, HSA was extracted from transgenic chloroplasts. FIG. 6(a) shows a silver stained SDS-PAGE gel in which HSA inclusion bodies could be separated from the soluble fraction (lane 3), where most of the cellular proteins are found. After solubilization of inclusion bodies and subsequent refolding, HSA could be completely converted into monomeric forms (FIG. 6a, lane 5; FIG. 6b, lane 5). Our estimations of HSA yields at the end of the protocol are about 20% of the initial quantities in leaves, although the reported protocol has been performed at the laboratory scale and may be further optimized for industrial production. Expression of HSA in transgenic plants has been estimated to be cost effective with levels of expression as low as 0.1 mg HSA/g fresh weight (Farran et al., 2002). The recoveries after solubilizing the inclusion bodies and refolding the HSA are about 0.25 mg HSA/g fresh weight (excluding soluble HSA in transgenic chloroplasts), which exceeds cost-effective estimations of biopharmaceutical industries.

As is understood in the art any of a number and variety of HSA is suitable for use in this invention. For purposes of illustration the Applicant provides a non limiting references to HSA, which fully characterized the HSA proteins and genes.

Specifically, Blondeau, K., H. Boze, G. Jung, G. Moulin, and P. Galzy. 1994. Physiological approach to heterologous human serum albumin production by Kluyveromyces lactis in chemostat culture. Yeast 10:1297-1303Medline; Swinkels, B. W., A. J. J. van Ooyen, and F. J. Bonekamp. 1993. The yeast Kluyveromyces lactis as an efficient host for heterologous gene expression. Antonie Leeuwenhoek 64:187-201; Fleer, R., P. Yeh, N. Amellal, I. Maury, A. Fournier, F. Bacchetta, P. Baduel, G. Jung, H. L'Hôte, J. Becquart, H. Fukuhara, and J. F. Mayaux. 1991. Stable multicopy vectors for high-level secretion of recombinant human serum albumin by Kluyveromyces yeast. Bio/Technology 9:968-975Medline, describe HSA in more detail.

The naturally occurring HSA gene has been sequenced and the sequence reported by Minghetti et al., J. Biol. Chem. 261:6747-6757 (1986). HSA genes have also been described in U.S. Pat. No. 5,648,243. These references are hereby incorporated by reference.

Experimental Procedures

Chloroplast expression vectors pLDAsdHSA was constructed by inserting the HSA 1.8 kb EcoRI /NotI fragment into the multiple cloning site of the pLD vector (Daniell et al., 1998; Daniell et al., 2001b; De Cosa et al., 2001; Guda et al., 2000; Kota et al., 1999). This fragment contains the mature HSA coding sequence preceded by a Shine-Dalgarno (GGAGG) (SEQ ID NO:2) and it has an ATG as the initiation codon. These sequences were introduced by using the primer: 5′-GGAGGCAACCATGGATGCACACAAGAGTGAAGG-3′ (SEQ ID NO:3). For the pLDApsbAHSA vector, the 204 bp sequence including the promoter and the psbA 5′UTR, was amplified by PCR using tobacco DNA as template. The following primers were used: 5′ CCGTCGACGTAGAGAAGTCCGTATT-3′ (SEQ ID NO:4) and 5′-GCCCATGGTAAAATCTTGGTTTATTTA-3′ (SEQ ID NO:5). The fusion with the HSA gene was made at the NcoI site placed at the 3′ end of the psbA 5′UTR and then inserted into the pLD vector as a EcoRI/ NotI fragment. Before proceeding with the bombardment, vectors were tested by Western blot analysis in E. coli. Bombardment and regeneration. Sterile tobacco (cv. Petit Havana) leaves were bombarded using the Bio-Rad PDS-1000/He biolistic device as described previously (Daniell, 1997). Bombarded leaves were subjected to two rounds of selection on the RMOP medium containing 500 mg/mL of spectinomycin to regenerate transformants (Daniell, 1997). After regeneration, plants were rooted on 500 mg/mL of spectinomycin (Daniell et al., 2001b) and transferred to pots in growth chambers. Photoperiod was 16 hours light and 8 hours dark. PCR and Southern blot analysis PCR was used to analyze integration of different cassettes in the transformed plants as described (Daniell et al., 2001b,c; De Cosa et al., 2001; Kota et al., 1999).

For Southern blot analysis, total DNA was extracted from leaves of transformed and untransformed plants (Qiagen Dneasy Kit). Total DNA (5 mg) was digested with BamHI, electrophoreses on 0.7% agarose gels and transferred to nylon membranes (Duralon-UV Stratagene). The template for probing flanking sequences was a 0.81 kb BglII/BamHI fragment and for HSA a 0.75 kb NcoI fragment. The probes were labeled with 32P-dCTP using the oligo-labelling procedure (Ready To Go, Amersham). Probes were hybridized to the membranes following the QUICK-HYB protocol (Duralon-UV, Stratagene).

Northern blot analysis Total RNA was extracted from leaves of transformed and untransformed plants (Rneasy Plant Kit, Qiagen). RNA 2.5 mg was electrophoreses on 1.2% agarose/formaldehyde gels and then transferred to nylon membranes (Stratagene). A 0.21 kb XbaI/PstI fragment of the 3′psbA gene was used as probe and labeled with 32P-dCTP using the oligo-labelling procedure (Amersham). HSA quantification. The ELISA Human Albumin Quantification Kit (Bethyl Laboratories) was used.

Transformed and untransformed leaves (100 mg) from potted plants grown under a 16 hours photoperiod were ground in liquid nitrogen, resuspended in 700 mL of 50 mM NaOH and analyzed following the manufacturer's protocol. Transgenic leaf extracts were diluted to fit in the linear range of the provided HSA standard. Absorbency was read at 450 nm. The DC protein assay (Bio-Rad) was used to determine total solubilized protein. SDS-PAGE and immunoblot analysis.

Transformed and untransformed leaves (100 mg) were ground in liquid nitrogen and resuspended in 200 mL of protein extraction buffer (200 mM Tris-HCl pH 8.0, 100 mM NaCI, 400 mM Sucrose, 14 mM bME, 0.05% Tween20, 0.1% SDS, 10 mM EDTA, 2 mM PMSF). Leaf extracts were boiled in sample buffer (Bio-Rad) and electrophoreses in a 10% polyacrylamide gel. Separated proteins were stained with Coomassie Brilliant Blue G-250 or transferred to a nitrocellulose membrane for immunoblotting, The primary antibody (rabbit anti-HSA, Nordic Immunology) was used at 1:10 000 dilution, and the secondary antibody (alkaline phosphatase conjugated mouse anti-rabbit, Sigma or goat anti-rabbit HRP conjugated, Southern Biotechnology) at 1:15 000.

Alkaline phosphatase color development reagents, BCIP/NBT, in AP Color Development Buffer (Bio-Rad) or the ECL kit (Amersham) were used for detection. Solubilization of inclusion bodies Soluble proteins were removed with a first extraction in 0.2 M NaCl, 25 mM Tris-HCl pH 7.4, 2 mM PMSF and 0.1% Triton X-100. After centrifugation for 60 min at 20 000 g, the pellet was solubilized for 16 h at 4 ° C. in 6 M Gu-HCl, 0.1 M bME and 0.25 mM Tris-HCl pH 7.4. After centrifugation for 60 min at 20 000 g, the supernatant was then slowly diluted 100-fold in 100 mM NaCl, 50 mM Tris-HCl pH 8.5 and 1 mM EDTA for 24 h at 4 ° C. Fractions were electrophoresed in a SDS-PAGE 10% gel and silver stained with Bio-Rad reagents and protocol.

Transmission electron microscopy and immunogold labeling Seedlings and mature leaves from untransformed and transgenic plants were analyzed. Fixation and immunogold labeled electron microscopy were performed as described by Vrekleij and Leunissen (1989). Sections were first blocked, incubated for 1 h with a goat antihuman albumin polyclonal antibody (Nordic Immunology; dilution range from 1:1000 to 1:10 000) and then incubated for 2 h with a rabbit anti-goatIgG secondary antibody conjugate to 10 nM gold diluted 1:40 in blocking solution. Sections were examined in a Zeiss EM 10 transmission electron microscope at 60 kV.

### REFERENCES

1. Bogorad, L. (2000) Engineering chloroplasts: an alternative site for foreign genes, proteins, reactions and products. Trends Biotechnol. 18, 257-263.

2. Carrio, M. M., Corchero, J. L. and Villaverde, A. (1999) Proteolytic digestion of bacterial inclusion body proteins during dynamic transition between soluble and insoluble forms. Biochim. Biophys. Act. 1434, 170176.

3. Daniell, H. (1997) Transformation and foreign gene expression in plants mediated by microprojectile bombardment. Meth. Mol. Biol. 62, 463-489.

4. Daniell, H. (2002) Molecular strategies for gene containment in transgenic crops. Nat. Biotechnol. 20, 581-586.

5. Daniell, H., Datta, R., Varmna, S., Gray, S. and Lee, S. B. (1998) Containment of herbicide resistance through genetic engineering of the chloroplast genome. Nat. Biotechnol. 16, 345-348.

6. Daniell, H. and Dhingra, A. (2002) Multigene engineering: Dawn of an exciting new era in biotechnology. Curr. Opin. Biotechnol. 13, 136-141.

7. Daniell, H., Dhingra, A. and Fernandez-San Millan, A. (2001a) Chloroplast transgenic approach for the production of antibodies, biopharmaceuticals and edible vaccines. Proceedings of the International Congress on Photosynthesis, S40-04. Brisbane, Australia. Colling wood, Australia: CSIRO Publishing.

8. Daniell, H., Khan, M. S. and Allison, L. (2002) Milestones in chloroplast genetic engineering: an environmentally friendly era in biotechnology. Trends Plant Sci. 7, 8491.

9. Daniell, H., Lee, S. B., Panchal, T. and Wiebe, P. O. (2001b) Expression of the native cholera toxin B subunit gene and assembly as functional oligomers in transgenic tobacco chloroplasts. J. Mol. Biol. 311, 1001-1009.

10. Daniell, H., Muthukumar, B. and Lee, S. B. (2001c) Marker free transgenic plants: Engineering the chloroplast genome without the use of antibiotic selection. Curr. Genet. 39, 109-116.

11. Daniell, H., Streatfield, S. and Wycoff, K. (2001d) Medical molecular farming: production of antibodies, biopharmaceuticals and edible vaccines in plants. Trends Plant Sci. 6, 219-226.

12. Daniell, H., Vivekananda, J., Neilsen, B., Ye, G. N., Tewari, K. K. and Sanford, J. C. (1990) Transient foreign gene expression in chloroplasts of cultured tobacco cells after biolistic delivery of chloroplast vectors. Proc. Natl Acad. Sci. USA, 87, 88-92.

13. De Cosa, B., Moar, W., Lee, S. B., Miller, M. and Daniell, H. (2001) Overexpression of the Bt cry2Aa2 operon in chloroplasts leads to formation of insecticidal crystals. Nat. Biotechnol. 19, 71-74.

14. DeGray, G., Rajasekaran, K., Smith, F., Sanford, J. and Daniell, H. (2001) Expression of an antimicrobial peptide via the chloroplast genome to control phytopathogenic bacteria and fungi. Plant Physiol. 127, 852-862.

15. Dodsworth, N., Harris, R., Denton, K., Woodrow, P., Wood, C. and Quirk, A. (1996) Comparative studies of recombinant human albumin and human serum albumin derived by blood fractionation. Biotechnol. Appl. Biochem. 24, 171-176.

16. Eibl, C., Zou, Z., Beck, A., Kim, M., Mullet, J. and Koop, H. U. (1999) In vivo analysis of plastid psbA, rbcL and rpl32 UTR elements by chloroplast transformation: tobacco plastid gene expression is controlled by modulation of transcript levels and translation efficiency. Plant J. 19, 333-345.

17. Enfors, S. O. (1992) Control of in vivo proteolysis in the production of recombinant proteins. Trends Biotechnol. 10, 310-315.

18. Farran, I., Sanchez-Serrano, J. J., Medina, J. F., Prieto, J. and Mingo-Castel, A. M. (2002) Targeted expression of human serum albumin to potato tubers. Transgenic Res. 11, 337-346.

19. Fleer, R., Yeh, P., Amellal, N., Maury, I., Fournier, A., Bacchetta, F., Baduel, P., Jung, G., Lhote, H., Becquart, J., Fukuhara, H. and Mayaux, J.F. (1991) Stable multicopy vectors for high-level secretion of recombinant human serum albumin by Kluyveromyces yeasts. Bio/Technol. 9, 968-975.

20. Guda, C., Lee, S. B. and Daniell, H. (2000) Stable expression of a biodegradable protein-based polymer in tobacco chloroplasts. Plant Cell Report, 19, 257-262.

21. Ketchner, S. L., Drapier, D., Olive, J., Gaudriault, S., Girard-Bascou, J. and Wollman, F. A. (1995) Chloroplasts can accomodate inclusion bodies. J. Biol. Chem. 270, 15299-15306.

22. Kota, M., Daniell, H., Varma, S., Garczynski, F., Gould, F. and Moar, W. J. (1999) Overexpression of the Bacillus thuringiensis (Bt) Cry2Aa2 protein in chloroplasts confers resistance to plants against susceptible and Bi-resistant insects. Proc. Natl Acad. Sci. USA, 96, 1840-1845.

23. Latta, L., Knapp, M., Sannientos, P., Brefort, G., Becquart, J., Guerrier, L., Jung, G. and Mayaux, J. (1987) Synthesis and purification of mature human serum albumin from E. coli. Bio/Technol. 5, 1309-1314.

24. Mayfield, S. P., Yohn, C. B., Cohen, A. and Danon, A. (1995) Regulation of chloroplast gene expression. Annu. Rev. Plant Physiol. Plant Mol. Biol. 46, 147-166.

25. Mitraki, A. and King, J. (1989) Protein folding intermediates and inclusion body formation. Bio/Technol. 7, 690-697.

26. Ohtani, W., Ohda, T., Sumi, A., Kobayashi, K. and Ohmura, T. (1998) Analysis of Pichia pastoris components in recombinant human serum albumin by immunological assays and by HPLC with pulsed amperometric detection. Anal. Chem. 70, 425-429.

27. Peters, T. (1995) The Albumin molecule. In: All About Albumin: Biochemistry, Genetics and Medical Applications. Chap. 6, pp. 251-284. San Diego, CA: Academic Press.

28. Petersen, C. E., Ha, C., Harohalli, K., Feix, J. B. and Bhagavan, N. V. (2000) A dynamic model for bilirrubin binding to human serum albumin. J. Biol. Chem. 275, 20985-20995.

29. Petrides, D., Sapidou, E. and Calandranis, J. (1995) Computer aided process analysis and economic evaluation for biosynthetic human insulin production—a study case. Biotechnol. Bioeng. 48, 529-541.

30. Quirk, A. V., Geisow, M. J., Woodrow, J. R., Burton, S. J., Wood, P. C., Sutton, A. D., Johnson, R. A. and Dodsworth, N. (1989) Production of recombinant human serum albumin from Saccharomyces cerevisiae. Biotechnol. Appl. Biochem. 11, 273-287.

31. Rozkov, A., Schweder, T., Veide, A. and Enfors, S. O. (2000) Dynamics of proteolysis and its influence on the accumulation of intracellular recombinant proteins. Enzyme Microbiol. Technol. 27, 743-748.

32. Ruf, S., Hermann, M., Berger, I. J., Carrer, H. and Bock, R. (2001) Stable genetic transformation of tomato plastids and expression of a foreign protein in fruit. Nat. Biotechnol. 19, 870-875.

33. Saunders, C. W., Schmidt, B. J., Mallonee, R. L. and Guyer, M. S. (1987) Secretion of human serum albumin from Bacillus subtilis. J. Bacteriol. 169, 2917-2925.

34. Shively, J. M. and English, R. S. (1991) The carboxysome, a prokaryotic organelle: a mini review. Can. J. Bot. 69, 957-962.

35. Sijmons, P. C., Dekker, B. M., Schrammeijer, B., Verwoerd, T. C., van den Elzen, P. J. and Hoekema, A. (1990) Production of correctly processed human serum albumin in transgenic plants. Bio/Technol. 8, 217-221.

36. Staub, J. M., Garcia, B., Graves, J., Hajdukiewicz, P. T., Hunter, P., Nehra, N., Paradkar, V., Schlittler, M., Carroll, J. A., Spatola, L., Ward, D. Ye, G. and Russell, D. A. (2000) High-yield production of a human therapeutic protein in tobacco chloroplasts. Nat. Biotechnol. 18, 333-338.

37. Staub, J. M. and Maliga, P. (1993) Accumulation of D1 polypeptide in tobacco plastid is regulated via the untranslated region of the psbA mRNA. EMBO J. 12, 601-606.

38. Staub, J. M. and Maliga, P. (1994) Translation of psbA mRNA is regulated by light via the 5′-untranslated region in tobacco plastids. Plant J. 6, 547-553.

39. Tarelli, E., Mire-Sluis, A., Tivnann, H. A., Bolgiano, B., Crane, D. T., Gee, C., Lemercinier, X., Athayde, M. L., Sutcliffe, N., Corran, P.H. and Rafferty, B. (1998) Recombinant human albumin as a stabilizer for biological materials and for the preparation of international reference reagents. Biologicals, 26, 331-346.

40. Vrekleij, A. J. and Leunissen, J. M., eds. (1989) Immuno-Gold Labeling in Cell Biology. Boca Raton, Fla.: CRC Press.

41. Watanabe, H., Kragh-Hansen, U., Tanase, S., Nakajou, K., Iwao, Y., Maruyama, T. and Otagiri, M. (2001a) Conforrnational stability and warfarin-binding properties of human serum albumin studied by recombinant mutants. Biochem. J. 357, 269-274.

42. Watanabe, H., Yamasaki, K., Kragh-Hansen, U., Tanase, S., Harada, K., Suenaga, A. and Otagiri, M. (2001b) In vitro and in vivo properties of recombinant human serum albumin from Pichia pastoris purified by a method of short processing time. Pharm. Res. 18, 1775-1781.

43. Zerges, W. (2000) Translation in chloroplasts. Biochimie, 82, 583601.

1. A plastid transformation vector for stably transforming a plastid genome comprising, as operably linked components, a first flanking sequence, a DNA sequence coding for psbA or a substantially homologous sequence of psbA, a 5′untranslated region (UTR), a DNA sequence coding for human serum albumin (HSA), and a second flanking sequence.

2. The vector of claim 1 further comprising a regulatory sequence.

3. The vector of claim 2, wherein said regulatory sequence comprises a promoter operative in said plastid.

4. The vector of claim 2, wherein said promoter is Prrn.

5. The vector of claim 1, wherein the transformation vector is competent for stabling integrating in the plastid genome of higher plant species and wherein the flanking sequences are substantially homologous to sequences in a spacer region of said plastid genome, and wherein said flanking sequences are conserved in the plastid genome of said higher plant species.

6. The vector of claim 5, wherein said spacer region is a transcriptionally active spacer region.

7. The vector of claim 1, wherein the plastid is selected from the group consisting of chloroplast, chromoplast, amyloplast, proplastid, leucoplast and etioplast.

8. The vector of claim 1, further comprising a DNA sequence encoding a selectable marker.

9. The vector of claim 8, wherein said selectable marker is an antibiotic- free selectable marker.

10. The vector of claim 9, wherein said DNA sequence encoding a selectable marker encodes Betaine aldehyde dehydrogenase (BADH).

11. The vector of claim 8, wherein said DNA sequence encoding a selectable marker encodes an antibiotic resistance selectable marker.

12. The vector of claim 11, wherein said antibiotic resistance selectable marker is aadA.

13. The vector of claim 1, wherein the 5′UTR is a 5′UTR of said psbA.

14. A plastid transformation vector for stably transforming a plastid comprising, as operably linked components, a first flanking sequence, a promoter operative in said plastid, a DNA sequence coding for a selectable marker capable of expression in said plastid, a DNA sequence coding for human serum albumin or a substantially homologous sequence thereof, and a second flanking sequence.

15. The vector of claim 1, competent for hyper-expression of HSA, in a plant transformed with said plastid transformation vector, wherein said plant expresses at least 0.1 mg HSA per gram/g fresh weight of said plant.

16. An isolated HSA, wherein said HSA is contained within inclusion bodies, and wherein said inclusion bodies are located in chloroplasts transformed with the vector of claim 1.

17. The isolated HSA of claim 16, wherein said inclusion bodies reduce the protolysis of HSA.

18. The isolated and purified HSA of claim 17, wherein said HSA is recovered from said inclusion bodies wherein said HSA maintains proper folding after the HSA is recovered from said inclusion bodies.

19. The isolated HSA of claim 18, wherein the HSA is properly refolded when removed from said inclusion bodies so the the HSA is structurally equivalent to native human HSA.

20. The isolated HSA of claim 19, wherein the inclusion bodies facilitate purification of said HSA from other cellular proteins.

21-44. (canceled)
